(19) United States
(12) Patent Application Publication
(10) Pub. No.: US 2015/0010987 A1
(43) Pub. Date: Jan. 8, 2015

(54) METHOD FOR PRODUCING EUGLENA HAVING HIGH WAX ESTER CONTENT

(71) Applicants: euglena Co., Ltd., Tokyo (JP); JX Nippon Oil & Energy Corporation, Tokyo (JP)

(72) Inventors: Ryo Arashida, Tokyo (JP); Yuka Marukawa, Tokyo (JP); Nobuo Aoki, Tokyo (JP); Hitoshi Matsuda, Tokyo (JP); Hiroaki Kato, Tokyo (JP); Akira Yoneda, Tokyo (JP)

(21) Appl. No.: 14/375,500

(22) PCT Filed: Jan. 31, 2013
(86) PCT No.: PCT/JP2013/052124

§ 371 Date: Jul. 30, 2014

(30) Foreign Application Priority Data
Jan. 31, 2012 (JP) JP2012-019026

(51) Int. Cl. C12N 1/12; C12P 7/64

(52) U.S. Cl. CPC C12N 1/12; C12P 7/6463
USPC 435/257.4

(57) ABSTRACT

Provided is a method that is for producing Euglena having a high wax ester content, capable of more efficiently producing the Euglena having a high wax ester content by adding a nutrient before anaerobic fermentation to restore the efficiency of wax ester fermentation. The present invention relates to a method for producing Euglena having a high wax ester content. The main configuration of the method is that there is at least a first step of aerobically culturing microalga Euglena under nitrogen starvation conditions and a second step of holding the cells in an anaerobic state; and adding, before the second step, a nutrient source to the culture liquid which has passed through the first step.

### TECHNICAL FIELD

The present invention relates to a method for producing Euglena having a high wax ester content, capable of producing microalga Euglena having a high content of wax esters that become a raw material for biofuels at low energy and low cost.

### BACKGROUND ART

In recent years in which global warming gains prominent attention, emission control of carbon dioxide gas that is one of greenhouse gases or reduction in atmospheric concentration of carbon dioxide by fixation of the carbon dioxide has become a significant issue.

Under such a situation, use of fossil fuels containing fixed carbon dioxide for energy leads to re-emission of the fixed carbon dioxide into the atmosphere, and brings about an environment issue. Further, the fossil fuels have a problem of depletion since they are limited resources.

To solve the problems as described above, fuel sources other than the fossil fuels are required, and expectations for development of biofuels made from higher plants and algae are being raised.

As possible higher plants as the biofuel material, Soybean, corn, palm and the like are known. However, the use of edible crops as the material is problematic from the point of concerns about food shortages.

On the other hand, although the production from non-edible plants such as Jatropha and Camelina is also promoted, it has the problem of low production volume per unit area.

Meanwhile, photosynthetic microorganisms and protozoans widely living in ponds or bogs have the same photosynthetic capability as the plants to biosynthesize carbohydrates or lipids from water and carbon dioxide, and accumulate several tens % by mass thereof in the cells. The production volume thereof is high, compared with the plants, and is known to be more than 10 times higher than palm that is said to have a high production volume per unit area.

Microalga Euglena, that is a photosynthetic microorganism, includes Euglena Ehrenberg that belongs to a group of flagellates and is famous as a motile alga. Most of Euglena have chloroplasts and live autotrophically through photosynthesis, but some are predacious or nutrient-absorptive. Euglena is a genus belonging to both zoology and botany.

Zoologically, Eulgenida is in an order belonging to Phytomastigophorea, Mastigophorea of Protozoa, and this consists of three suborders of Euglenoidina, Peranemoidina, and Petalomonadoidina.

Euglenoidina includes, as genera, Euglena, Trachelemonas, Strombonas, Phacus, Lepocinelis, Astasia, and Colacium. Botanically, Euglenales belongs to Euglenophyceae of Euglenophyta, and this order includes the same genera as in the zoological classification, in addition to Euglena.

Euglena accumulates paramylon in the cells as carbohydrate.

Paramylon is a particle of a macromolecule in which about 700 glucoses are polymerized by β-1,3-bond.

Euglena performs, when placed in an anaerobic state, wax ester fermentation to decompose paramylon that is a storage polysaccharide and produce wax esters each composed of a fatty acid and a fatty alcohol as a final product.

The components of vegetable oils and fats, included in general algae, correspond to gas oil having a carbon number distribution of main framework of 16 or more, or petroleum fractions heavier than them, while the wax esters of Euglena are composed of fatty acids and alcohols having a carbon number of about 14. This shows that biomass fuels obtained from the wax esters of Euglena are within the carbon number distribution range of existing jet fuels of 10 to 16, and can be easily purified into jet fuels, compared with other vegetable oils and fats, in production of hydrocarbons by fuelization (hydrogenation, isomerization).

Under such circumstances, techniques for producing clean and useful energy by performing wax ester fermentation using microorganisms have been searched.

Patent Document 1 describes that storage polysaccharide paramylon is fermented and converted into wax esters by aerobically culturing Euglena and then maintaining it under anaerobic conditions.

### CITATION LIST

### Patent Document

Patent Document 1: JPH 0365948 B2

### SUMMARY OF THE INVENTION

### Problems to be Solved by the Invention

Although to produce energy through wax ester fermentation using microorganisms has been proposed as seen above, the technique of Patent Document 1 discloses, as an aerobic culture method, nothing but a general method such as addition of organic matter such as glucose as a carbon source or culture under general photosynthetic conditions.

In this technique, the culture method using the carbon source such as glucose is not worth the cost for the production of biofuels, and it does not lead to the fixation of carbon dioxide.

Other techniques include Japanese Patent Application No. 2010-163370 presented in the past by the applicant (a technique which was undisclosed when the present application was filed).

This discloses a method for producing Euglena having a high wax ester content, comprising a first step of aerobically culturing microalga Euglena under autotrophic culture conditions with introduction of carbon dioxide, a second step of further culturing the cells in a nitrogen-starved state to increase the accumulation amount of paramylon per cell; and a third step of holding the cells in an anaerobic state to perform wax ester fermentation using paramylon as the substrate.

Namely, in this technique, a sequence of steps of aerobic culture, additional culture in nitrogen-starved state and holding cells in anaerobic state are carried out, whereby Euglena having a high content of wax esters can be efficiently produced.

In this technique, carbohydrates can be sufficiently accumulated in the Euglena by the culture in nitrogen-starved state of Step 2.

Since the carbohydrates sufficiently accumulated in Step 2 are converted into wax esters in Step 3 by placing the cells cultured in Step 2 in the anaerobic state, the accumulation amount of wax esters in Step 3 is consequently increased.

According to this technique, Euglena having a high wax ester content can be surely produced.

However, when the cells are placed in the nitrogen-starved state for a long period of time, the efficiency of the wax ester fermentation is deteriorated. Since the enzyme related to the fermentation is a protein, a nitrogen source for biosynthesizing amino acids constituting the protein is needed. However, in the nitrogen-starved state, additional supply of the nitrogen source from the outside of the cells is stopped. Namely, a reduction in production volume of the enzyme related to the fermentation in the Euglena cells is considered to lead to the reduction in fermentation efficiency.

To solve each of the above-mentioned problems, the present invention thus has an object to provide a method for producing Euglena having a high wax ester content, capable of more efficiently producing the Euglena having a high wax ester content by adding a nutrient before anaerobic fermentation to restore the efficiency of wax ester fermentation.

### Means for Solving the Problems

The above-mentioned problem can be solved by a method for producing Euglena having a high wax ester content of the present invention, the method comprising performing at least a first step of aerobically culturing microalga Euglena under nitrogen starvation conditions and a second step of holding the cells in an anaerobic state, and adding, before the second step, a nutrient source to the culture liquid which has passed through the first step.

In this way, before the second step, the nutrient source is added to the culture liquid which has passed through the first step, in addition to a sequence of steps of aerobic culture and holding cells in anaerobic state, whereby the Euglena having a high wax ester content can be efficiently produced.

Namely, carbohydrates can be sufficiently accumulated in the Euglena by the culture in nitrogen-starved state that is the first step.

Therefore, the cultured cells are placed in the anaerobic state in the second step, whereby the sufficiently accumulated carbohydrates can be converted into the wax esters.

However, when the cells are placed in the nitrogen-starved state for a long period of time, the supply of the nitrogen source which contributes to the biosynthesis of the amino acids constituting the enzyme related to the fermentation is stopped, resulting in reduced efficiency of the wax ester fermentation.

Namely, the biosynthesis amount of the protein constituting the enzyme is reduced, resulting in reduced fermentation efficiency.

If the Euglena cultured in the first step is used and transferred to the second step as it is, the production efficiency of the wax esters in the anaerobic fermentation of the second step is reduced to keep the ratio of wax esters to diglyceride and triglyceride at a low level, although the accumulation amount of paramylon that becomes the raw material for the wax esters increases.

Accordingly, the nutrient source is added, before the anaerobic fermentation is performed in the second step, to suppress the reduction in fermentation efficiency in the second step, whereby Euglena having a higher wax ester content can be efficiently produced, and the ratio of wax esters to diglyceride and triglyceride can be secured at high level so as to be suitable for the production of fuel oil bases for aviation fuel.

The addition of the nutrient is preferably performed at earlier timing, based on the point of time at which the dissolved oxygen concentration of the culture liquid in the anaerobic state falls below 0.03 mg/L in the second step.

Since the nitrogen starvation state is dissolved by the addition of the nutrient, decomposition of the accumulated paramylon is promoted.

Therefore, if the timing of adding the nutrient is too early, the nitrogen starvation state is restored due to the consumption of the added nutrient during the culture.

In this way, the timing of adding the nitrogen source is important, and is preferably controlled by time based on the point of time at which the dissolved oxygen concentration of the culture liquid in the anaerobic state falls below 0.03 mg/L.

Concretely, the addition of the nutrient source is performed preferably 3 hours before anaerobic fermentation, more preferably within 1 hour before anaerobic fermentation.

Specifically, the nutrient source is preferred to be a nitrogen source.

The nutrient source may be a carbon source, and combined use of the nitrogen source and the carbon source is more preferred.

As the “nitrogen source”, an ammonium compound such as diammonium hydrogenphosphate or ammonium sulfate or an amino acid such as glycine or glutamate is preferably selected.

When Euglena, which generally cannot assimilate nitrate nitrogen, is modified by a genetic recombination technique or the like so that nitric acid can be assimilated, nitrate nitrogen absorbed from the outside of the cells is supposed to be metabolized by ammonia nitrogen. If such is the case, a nitric acid compound can be included in the possible nitrogen sources.

As the “carbon source”, a glucide such as glucose or fructose, an alcohol such as ethanol, an organic compound such as malic acid, or an amino acid such as glutamate is preferably selected.

From the viewpoint of advantages of yield, availability, cost and the like, it is preferred to select the ammonium compound as the nitrogen source and glucose as the carbon source.

In addition to the above-mentioned conditions, the amount of the nutrient source being added is also important.

An excessively large addition amount of the nutrient source causes a reduction in the accumulation amount of paramylon, and an excessively small addition amount does not lead to the improvement in anaerobic fermentation efficiency.

Therefore, in the present invention, the ammonium compound is preferably added so that the ammonium ion concentration becomes about 10 mg/L.

As described above, to solve the above-mentioned problems, the method for producing Euglena having a high wax ester content of the present invention is most characterized by performing, in execution of a culture step of culturing Euglena in nitrogen-starved state to accumulate carbohydrates and an anaerobic fermentation step of placing the cultured cells in an anaerobic state to convert the carbohydrates to wax esters, addition of a nutrient source before the anaerobic fermentation step.

### Effect of the Invention

According to the present invention, a sequence of steps of sufficiently accumulating carbohydrates in Euglena through the culture in nitrogen-starved state, and converting the sufficiently accumulated carbohydrates into wax esters by placing the cultured cells in the anaerobic state is performed, and during these steps, the fermentation efficiency of the wax esters is restored by adding the nutrient before the anaerobic fermentation, whereby Euglena having a high wax ester content can be more effectively produced.

Therefore, biomass raw materials high in fat and oil content can be inexpensively produced from carbon dioxide fixed by photosynthesis.

The production of biofuels by the present invention leads to an improvement in energy self-sufficiency rate.

### BRIEF DESCRIPTION OF THE DRAWINGS

FIG. 1 is a process chart showing a method for producing Euglena having a high wax ester content according to one embodiment of the present invention.

FIG. 2 is a chart showing a result of GPC analysis for Comparative Example 1 of the present invention.

FIG. 3 is a chart showing results of GPC analysis for Example 1 of the present invention.

FIG. 4 is a chart showing results of GPC analysis for Example 2 of the present invention.

FIG. 5 is a chart showing results of GPC analysis for Example 3 of the present invention.

### DETAILED DESCRIPTION OF THE INVENTION

One embodiment of the present invention will be then described in reference to the drawings.

The configuration to be described below never limits the present invention and can be variously modified without departing from the gist of the present invention.

This embodiment relates to a method for producing Euglena by culturing Euglena under aerobic conditions and then placing the cells in an anaerobic state, the method capable of more effectively producing Euglena having a high wax ester content by adding a nutrient before the anaerobic fermentation to restore the fermentation efficiency of wax esters.

### First Embodiment

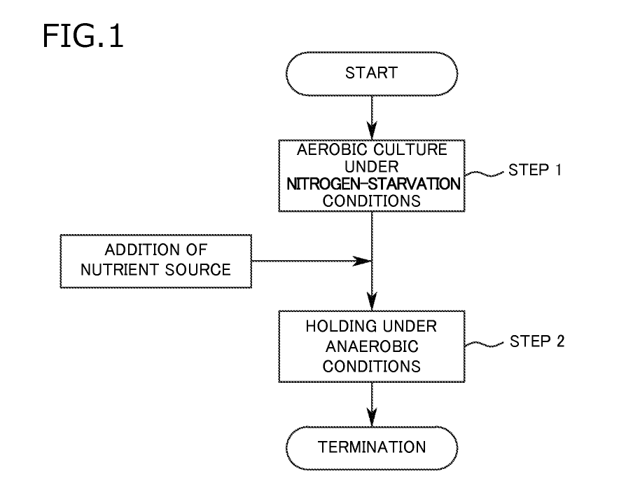

A first embodiment of the method for producing Euglena having a high wax ester content of the present invention will be described in reference to FIG. 1.

This method comprises: Step 1 (corresponding to the first step) of aerobically culturing Euglena in a medium under nitrogen starvation conditions; and Step 2 (corresponding to the second step) of fermenting carbohydrates into wax esters through an anaerobic treatment.

Prior to Step 1 (corresponding to the first step), preculture of Euglena is performed.

An AY medium was used for the preculture.

The AY medium that is an autotrophic culture medium is preferably set to an acidic condition and, for example, is adjusted to pH 2.5 to 6.5, more preferably pH 3.0 to 6.0.

Specifically, in this embodiment, an AY medium having a composition shown in Table 1 was prepared using deionized water, adjusted to pH 3.5 using diluted sulfuric acid, and then subjected to autoclave sterilization.

The AY medium means an autotrophic culture medium obtained by removing heterotrophic components such as glucose, malic acid and amino acid from a Koren-Hutner medium which is generally used as a heterotrophic culture medium of Euglena.

Table 1 is one example of the autotrophic culture medium, wherein VB₁ represents vitamin B₁, and VB₁₂ represents vitamin B₁₂.

TABLE 1

| Component | g/L | Component | mg/L |
|---|---|---|---|
| (NH₄)₂SO₄ | 0.5 | FeSO₄•7H₂O | 50 |
| (NH₄)₂HPO₄ | 0.25 | MnCl₂•4H₂O | 18 |
| KH₂PO₄ | 0.25 | ZnSO₄•7H₂O | 25 |
| MgCO₃ | 0.6 | (NH₄)₆Mo₇O₂₄•4H₂O | 4 |
| CaCO₃ | 0.12 | CuSO₄ | 1.2 |
| MgSO₄•7H₂O | 0.6 | H₃BO₃ | 0.6 |
| | | VB₁ | 2.5 |
| | | VB₁₂ | 0.005 |

About 2 L of the sterilized AY medium was put in an acrylic culture vessel of 10 cm×10 cm×27 cm so as to have a water depth of 20 cm, and Euglena gracilis strain Z was planted therein.

The culture vessel was set in a constant-temperature water tank placed on a magnetic stirrer SRSB10LA (ADVANTEC), and stirred at a strength of 300 rpm by use of a 6-cm stirring bar.

Introduction of CO₂ is preferably performed at a flow rate of 0.05 vvm to 0.2 vvm (100 to 200 mL/min) under a light intensity of 600 to 1200 μmol/(m²·s).

The “vvm” represents “gas introduction volume per unit volume (volume per volume minute)”.

Specifically, in this embodiment, a metal halide lamp Eye Clean Ace BT type (Iwasaki Electric) was installed just above the surface of culture liquid surface while adjusting the height so that the light radiated onto the culture liquid surface had an intensity of about 900 μmol/(m²·s).

As the light irradiation time, a light-dark cycle of extinction for 12 hours after lighting for 12 hours was adopted to approximate outdoor day-and-night conditions. As the carbon source, 15%-concentration CO₂ was introduced at a flow rate of 0.1 vvm (200 mL/min).

The preculture time is set to 24 to 120 hours, preferably to 48 to 96 hours.

Similarly, the culture temperature is set preferably to 26 to 32° C., more preferably to 28 to 30° C.

Specifically, in this embodiment, after the preculture in the AY medium was performed for 3 days, the Euglena cells were centrifugally separated (2,500 rmp, 5 min., room temperature) from 2 L of the culture liquid, and then washed with deionized water once to prepare a seed alga body for each culture.

In Step 1 (corresponding to the first step), Euglena is aerobically cultured under nitrogen starvation conditions to increase the accumulation amount of paramylon.

A nitrogen starvation AY medium is preferably set to an acidic condition, and, for example, is adjusted to pH 2.5 to 6.5, more preferably to pH 3.0 to 6.0.

Specifically, in this embodiment, a nitrogen starvation AY medium having a composition shown in Table 2 is prepared using deionized water, adjusted to pH 3.5 using diluted sulfuric acid, and then subjected to autoclave sterilization.

The nitrogen starvation medium means a culture medium which has a nitrogenous compound content of 5 mg/L or less.

TABLE 2

| Component | g/L | Component | mg/L |
|---|---|---|---|
| KH₂PO₄ | 0.25 | FeSO₄•7H₂O | 50 |
| MgCO₃ | 0.6 | MnCl₂•4H₂O | 18 |
| CaCO₃ | 0.12 | ZnSO₄•7H₂O | 25 |
| MgSO₄•7H₂O | 0.6 | (NH₄)₆Mo₇O₂₄•4H₂O | 4 |
| | | CuSO₄ | 1.2 |
| | | H₃BO₃ | 0.6 |
| | | VB₁ | 2.5 |
| | | VB₁₂ | 0.005 |

About 4.5 L of the sterilized nitrogen starvation AY medium was put in an acrylic culture vessel of 15 cm×15 cm×27 cm so as to have a water depth of 20 cm, and the seed alga body precultured in the AY medium was planted therein.

The initial concentration of Euglena is set preferably to 0.05 to 5.0 g/L, more preferably to 0.2 to 1.0 g/L.

Specifically, in this embodiment, the culture is performed at an initial concentration of about 0.3 g/L and under culture conditions for light irradiation, stirring, aeration and the like which are the same ranges and methods as the preculture.

The nitrogen starvation culture time is set to within 48 hours (light period: within 24 hours).

In this embodiment, 48 hours was adopted.

This selection of the nitrogen starvation culture period will be described in detail in the following “Comparative Example 1”.

The light irradiation was carried out in a light-dark cycle of lighting a metal halide lamp after 12 hours, with the start of dark period being at 0 hour into the culture, turning off after 24 hours, and relighting after 36 hours.

In Step 2 (corresponding to the second step), anaerobic treatment of the cultured Euglena is performed, and the resulting Euglena is held in an anaerobic state.

The culture liquid is condensed from 2 L to about 0.5 L by use of a centrifugal separator, and put in a 600-mL tall beaker.

With respect to about 400 mL of the culture liquid, the anaerobic treatment is performed while introducing nitrogen gas at a flow rate of 200 mL/min for about 30 minutes.

The anaerobic treatment is terminated after confirming that the dissolved oxygen concentration falls below 0.03 mg/L.

The beaker after the introduction of nitrogen gas was entirely covered with an aluminum foil for light shielding after covering the upper portion thereof with a parafilm, and was allowed to stand still at room temperature for 3 days.

The room temperature is 26 to 27° C.

Although the anaerobic treatment is performed, in general, by introducing an inert gas such as nitrogen gas or argon gas to the culture medium after the culture as described above, the dissolved oxygen concentration can be reduced also by a treatment such as increasing the cell density by condensation of the culture liquid.

The anaerobic treatment can be performed also by allowing the medium to stand still.

Namely, when the medium is allowed to stand still without stirring, the cells are settled out and the density is increased, so that a lack of oxygen is caused. The anaerobic treatment may be performed by creating a high-density state by centrifugal separation.

The pH in this case may be any value except an extremely low or high value, and the presence or absence of light irradiation never affects the wax ester fermentation. The holding temperature may be any temperature except a high temperature such that Euglena is killed and a low temperature such that the medium is frozen. In general, the wax ester fermentation is completed in 6 to 72 hours.

In this embodiment, before performing Step 2, or before performing the anaerobic treatment, a nutrient source is added to the Euglena culture liquid.

The “before anaerobic treatment (before anaerobic fermentation)” means earlier timing, based on the point of time at which the dissolved oxygen concentration of the Euglena culture liquid falls below 0.03 mg/L.

As the nutrient source, a nitrogen source, a carbon source, a mixture of nitrogen source and carbon source and the like are conceivable.

The addition amount of the nitrogen source as the nutrient source is, in terms of ammonium ions, 7 to 15 mg/L, preferably 8 to 12 mg/L, relative to a treatment object liquid (the culture liquid obtained in Step 1).

The addition amount of the carbon source (glucose) as the nutrient source is 0.2 to 2.0 g/L, preferably 0.5 to 1.5 g/L, relative to a treatment object liquid (the culture liquid obtained in Step 1).

The nitrogen source includes an ammonium compound such as diammonium hydrogenphosphate or ammonium sulfate and an amino acid such as glycine or glutamate.

When Euglena, which generally cannot assimilate nitrate nitrogen, is modified by a genetic recombination technique or the like so that nitric acid can be assimilated, nitrate nitrogen absorbed from the outside of the cells is supposed to be metabolized by ammonia nitrogen. When this is the case, a nitric acid compound is also included in the nitrogen source.

The “carbon source” includes a glucide such as glucose or fructose, an alcohol such as ethanol, an organic compound such as malic acid, and an amino acid such as glutamate.

In this embodiment, the ammonium compound is used as the nitrogen source, and glucose is used as the carbon source.

When the nitrogen source is added, diammonium hydrogenphosphate ((NH₄)₂HPO₄) is added as the nitrogen source in an amount of 0.1643 g per L of the culture liquid (corresponding to 10 mg/L), at 47 hours into the nitrogen starvation culture, which is 1 hour before anaerobic treatment.

This timing of adding the nitrogen source will be described in detail in the following “Example 1”.

When the carbon source is added, glucose is added as the carbon source in an amount of 1 g per L of the culture liquid, at 47 hours into the nitrogen starvation culture, which is 1 hour before anaerobic treatment.

This timing of adding the carbon source will be described in detail in the following “Example 2”.

Further, when the nitrogen source and the carbon source are added in combination, glucose and diammonium hydrogenphosphate are added at 47 to 48 hours into the nitrogen starvation culture, which is 0 to 1 hour before anaerobic treatment.

This timing of adding the nitrogen source and the carbon source will be described in detail in the following “Example 3”.

The addition of the nutrient source is combined with the processes of Step 1 to Step 2, whereby an advantageous effect which cannot be obtained by simply performing each step, or a remarkable increase in accumulation amount of wax esters, can be attained.

Further, wax esters offering high evaluations also in qualitative evaluation of fat and oil composition can be produced.

The selection reason of each condition will be described in the following Comparative Example and Examples.

### Comparative Example 1

### Examination on Nitrogen Starvation Culture Period

(1) Preculture, Nitrogen starvation Culture, and Anaerobic Treatment

Preculture is the same as above.

After preculturing in the AY medium for 3 days, Euglena cells were centrifugally separated (2,500 rpm, 5 min., room temperature) from 2 L of culture liquid, and washed with deionized water once to prepare a seed alga body for nitrogen starvation culture.

A nitrogen starvation AY medium having the composition shown in the above-mentioned Table 2 was prepared using deionized water, and nitrogen starvation culture was performed in the same manner as the above-mentioned Step 1.

The culture was carried out in a light-dark cycle of lighting a metal halide lamp after 12 hours, with the start of dark period being at 0 hour into the culture, turning off after 24 hours, and relighting after 36 hours. The prepared culture liquid was taken as Sample 1-1.

Sample 1-1 was recovered at 48 hours into the culture.

An anaerobic treatment was performed in the same manner as the above-mentioned Step 2.

The Euglena cells were recovered from the culture liquid after the anaerobic treatment by centrifugal separation (2,500 rpm, 5 min., room temperature), and the recovered sediment was frozen followed by freeze-drying to obtain the following specimen.

The freeze-drying was performed by use of a freeze dryer DRW 240DA (Advantec).

### (2) Quantitative Determination of Carbohydrates

The carbohydrate content of Euglena dry powder was determined by the following method.

This determination is considered to substantially correspond to quantitative determination of paramylon, since about 90% of the carbohydrates contained in Euglena cells are paramylon.

About 0.1 g of dry Euglena powder was put in a 50-mL falcon centrifugal tube, and 10 mL of acetone was added thereto.

The mixture was fragmented for 90 seconds by an ultrasonic disintegrator (Tomy, UD-201) followed by centrifugal separation (2,000 rpm, 5 min., room temperature).

After supernatant liquid was poured off, the sediment was subjected to ultrasonic fragmentation and centrifugal separation under the above-mentioned conditions with 10 mL of acetone being added thereto.

After supernatant liquid was poured off again, the sediment was stirred and suspended by a vortex mixer with 20 mL of 1% SDS solution being added thereto, and then warmed in a boiled water bath for 30 minutes.

After the resultant was subjected to centrifugal separation (2,000 rpm, 5 min., room temperature), the centrifugal sediment was stirred and suspended by a vortex mixer with 10 mL of 0.1% SDS solution being added thereto.

After the resultant was subjected again to centrifugal separation (2,000 rpm, 5 min., room temperature), the centrifugal sediment was stirred and suspended by a vortex mixer with 20 mL of RO water being added thereto, and the sediment was washed.

The sediment, after being subjected to centrifugal separation (2,000 rpm, 5 min., room temperature), was suspended and dissolved in 20 mL of 0.5 N NaOH, and an extract, which was obtained from the suspension liquid allowed to stand still for several hours to one night, was subjected to sugar determination.

The sugar determination of the extract was performed by a phenol sulfuric acid method.

To 0.5 mL of an extract solution, 0.5 mL of 5% phenol and 2.5 mL of sulfuric acid were added, and the mixture was suspended by a vortex mixer.

After the resultant was allowed to stand still at room temperature for 20 to 30 minutes, absorbance at 480 nm thereof was read by a spectrophotometer (SHIMADZU, UVmini-1240).

For formation of calibration curves, a glucose solution (0 μg/mL, 10 μg/mL, 50 μg/mL, 150 μg/mL, 250 μg/mL) or a 0.005% paramylon solution was used.

### (3) Extraction and Quantitative Determination of Fat and Oil

The extraction and quantitative determination of fat and oil from the Euglena dry powder were performed by the following method.

Into a sealed vessel, 0.2 to 0.3 g of Euglena dry alga body was put, and shaken at room temperature and 200 rpm for 1 hour with 10 times its volume of n-hexane being added thereto.

Solid-liquid separation was performed by filtration, and the cake on the funnel was washed using about 20 times its original dry weight of hexane.

The filtrate and the wash liquid were joined together, and n-hexane was distilled therefrom by an evaporator which was set to a bath temperature of 55° C., whereby fat and oil were recovered.

The above-mentioned operation was repeated twice, and the first and second extracted oils and fats were joined together.

From the weight of the recovered oil and the weight of the Euglena dry alga body used for the hexane extraction, the content of fat and oil in the dry alga body after anaerobic treatment was calculated.

Although the weight per cell is reduced in the anaerobic fermentation of Euglena since CO₂ is released in the process of producing acetyl-CoA from pyruvic acid, no correction by weight reduction before and after anaerobic treatment is performed in Table 1.

### (4) Component Analysis of Fat and Oil

To examine the components of fat and oil extracted from Euglena dry powder, gel permeation chromatography (GPC) analysis was performed according to the following method.

A fat and oil dry solid after hexane extraction was dissolved by adding 10 mL of chloroform thereto, and then filtered to prepare a measurement solution.

As an HPLC system, Allience 2695 (Waters) was used, and as a column, G2000H8 (Tosoh) was used.

The measurement was carried out under conditions of column temperature: 23° C., flow velocity: 1 mL/min, concentration: 1.0 mass %, and injection amount: 100 μL, and as a detector, RI was used.

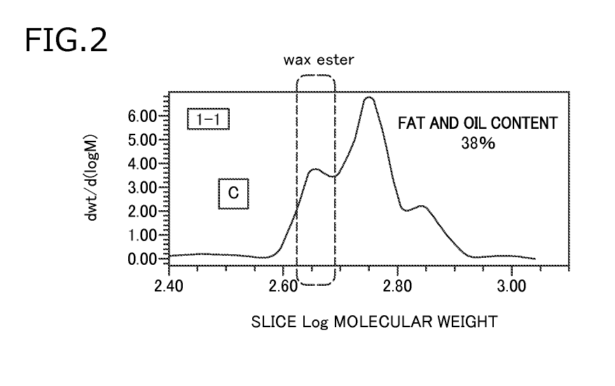

The results of this GPC analysis are shown in FIG. 2.

The quantitative determination results of carbohydrates before and after anaerobic treatment, the fat and oil contents before and after anaerobic treatment, and the qualitative evaluation of fat and oil composition are shown in Table 3.

TABLE 3

| | | Carbohydrate | | Fat and Oil | | Qualitative |
|---|---|---|---|---|---|---|
| | | Content [%] | | Content [%] | | Evaluation of |
| Sample | Examination | Before | After | Before | After | Fat and Oil |
| Name | Condition | Anaerobic | Anaerobic | Anaerobic | Anaerobic | Composition |
| 1-1 | Nitrogen | 49 | 12 | Not | 38 | C |
| | starvation | | | determined | | |
| | culture | | | | | |
| | period 48 h | | | | | |

It was found from the above-mentioned results that the content of carbohydrates is increased when the nitrogen starvation culture period is 48 hours (Sample 1-1).

Accordingly, it can be determined that a nitrogen starvation culture period of within 48 hours (bright period: within 24 hours) is preferred, considering accumulation of carbohydrates.

On the other hand, in Sample 1-1, the carbohydrate content is seriously reduced after anaerobic treatment, compared with before anaerobic treatment. This is consistent with the knowledge that most of the carbohydrates contained in Euglena are paramylon, and the paramylon is decomposed by anaerobic treatment.

With respect to the fat and oil composition, also, a satisfactory result was observed in Sample 1-1, as shown by the peak of wax ester of FIG. 2.

When the nitrogen starvation culture period is prolonged, the peak of wax ester becomes small.

This is attributed to that new amino acids are hardly biosynthesized within the Euglena cells due to nitrogen starvation stress to reduce the expression or protein translation volume of the gene coding the enzyme related to the anaerobic fermentation or to deteriorate the enzyme activity.

Taking into consideration this knowledge, a nitrogen starvation period exceeding 48 hours is considered to have no great effect, and the following comparative experiments were thus performed with the nitrogen starvation culture period being set to 48 hours.

### Example 1

### Examination on Addition of Nitrogen Source

(1) Preculture, Nitrogen starvation Culture and Anaerobic Treatment

It was found from the results of Comparative Example 1 that the nitrogen starvation culture period is enough to be within 48 hours in the viewpoint of carbohydrate accumulation.

On the other hand, the qualitative evaluation of fat and oil composition of C shows room for improvement.

Therefore, based on Sample 1-1 of Comparative Example 1, an experiment was carried out with respect to addition of a nitrogen source to culture liquid several hours before anaerobic treatment.

The conditions of preculture, nitrogen starvation culture and anaerobic treatment were the same as in Comparative Example 1.

### (2) Addition of Nitrogen Source

In this example, the nitrogen source is added before anaerobic treatment.

In Sample 2-1, diammonium hydrogenphosphate ((NH₄)₂HPO₄) was added, as the nitrogen source, in an amount of 0.1643 g (corresponding to 10 mg/L) per L of culture liquid, at 47 hours into the nitrogen starvation culture, which is 1 hour before anaerobic treatment.

Similarly, in Sample 2-2, diammonium hydrogenphosphate ((NH₄)₂HPO₄) was added, as the nitrogen source, in an amount of 0.1643 g (corresponding to 10 mg/L) per L of culture liquid, at 36 hours into the nitrogen starvation culture, which is 12 hours before anaerobic treatment.

### (3) Quantitative Determination of Carbohydrates, Extraction and Quantitative Determination of Fat and Oil, and GPC Analysis

These analyses were performed in the same conditions as in Comparative Example 1.

The fat and oil contents before and after anaerobic treatment are shown in Table 4.

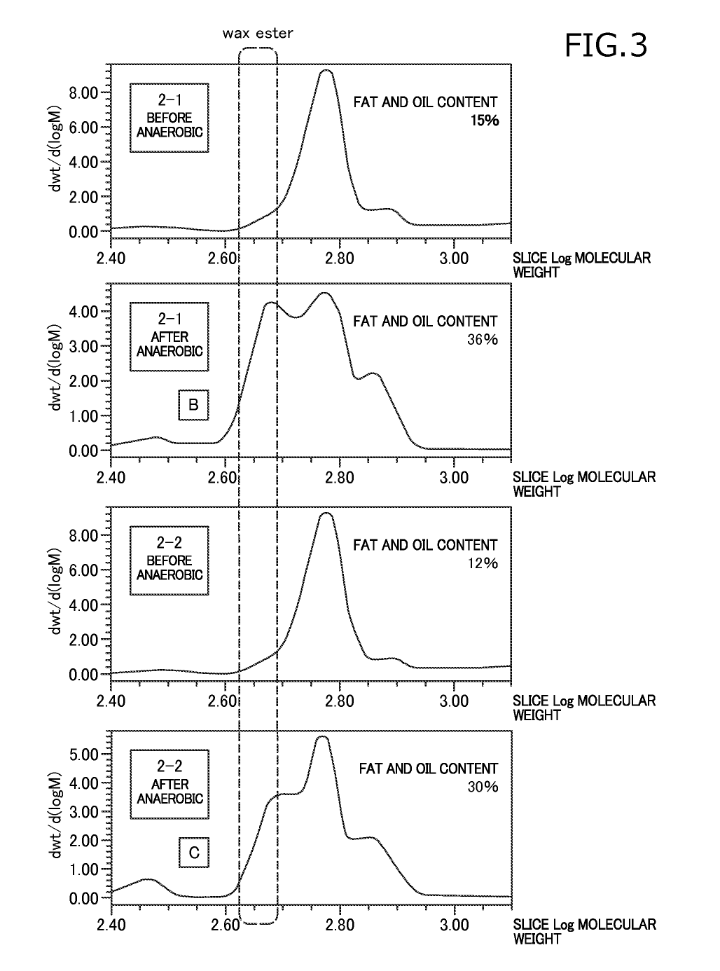

The result of GPC analysis is shown in FIG. 3.

TABLE 4

| | | Carbohydrate | | Fat and Oil | | Qualitative |
|---|---|---|---|---|---|---|
| | | Content [%] | | Content [%] | | Evaluation of |
| Sample | Examination | Before | After | Before | After | Fat and Oil |
| Name | Condition | Anaerobic | Anaerobic | Anaerobic | Anaerobic | Composition |
| 2-1 | Addition of N | 27 | 1 | 15 | 36 | B |
| | source 1 h | | | | | |
| | before anaerobic | | | | | |
| 2-2 | Addition of N | 23 | 2 | 12 | 30 | C |
| | source 12 h | | | | | |
| | before anaerobic | | | | | |

The addition of diammonium hydrogenphosphate 12 hours before anaerobic treatment resulted in a low fat and oil content, compared with Sample 1-1, with the fat and oil content being 30% and the qualitative evaluation of fat and oil composition being C, and had no effect (refer to Sample 2-2 of Table 4).

On the other hand, the addition of diammonium hydrogenphosphate 1 hour before anaerobic treatment had an improvement effect of fat and oil composition, with the fat and oil content being 38% and the qualitative evaluation of fat and oil composition being B, although the fat and oil content was slightly reduced, compared with Sample 1-1 (refer to Sample 2-1 of Table 4).

This is attributed to that, although a prolonged nitrogen starvation culture period leads to a reduction in the expression or protein translation volume of the gene coding the enzyme related to anaerobic fermentation, the expression and translation of the gene coding the enzyme related to anaerobic fermentation are promoted by the addition of the nitrogen source before anaerobic treatment, resulting in restored anaerobic fermentation capability.

### Example 2

### Examination on Addition of Carbon Source

### (1) Preculture, Nitrogen Starvation Culture, and Anaerobic Treatment

It was found from the results of Comparative Example 1 that the nitrogen starvation culture period is enough to be within 48 hours in the view of carbohydrate accumulation.

It was also found from the results of Example 1 that the anaerobic fermentation capability of Euglena cells is restored by adding the nitrogen source before anaerobic treatment.

In this example, an experiment on addition of glucose was carried out to examine whether the anaerobic fermentation capability is restored by adding not the nitrogen source but a carbon source.

The conditions of preculture, nitrogen starvation culture and anaerobic treatment were the same as in Comparative Example 1.

### (2) Addition of Carbon Source

In this example, the carbon source is added before anaerobic treatment.

In Sample 3-1, glucose was added as the carbon source in an amount of 1 g per L of culture liquid, at 47 hours into the nitrogen starvation culture, which is 1 hour before anaerobic treatment.

(3) Quantitative Determination of carbohydrate, Extraction and Quantitative Determination of Fat and Oil, and GPC Analysis

These analyses were performed in the same conditions as in Comparative Example 1.

The fat and oil contents before and after anaerobic treatment are shown in Table 5.

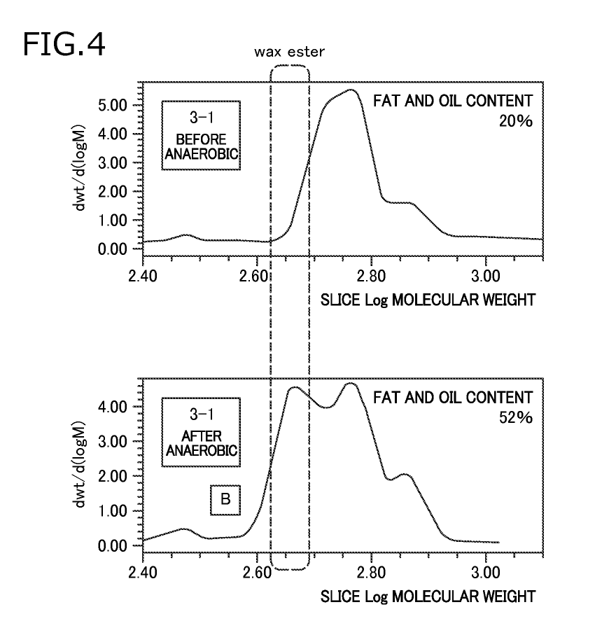

The result of GPC analysis is also shown in FIG. 4.

TABLE 5

| | | Carbohydrate | | Fat and Oil | | Qualitative |
|---|---|---|---|---|---|---|
| | | Content [%] | | Content [%] | | Evaluation of |
| Sample | Examination | Before | After | Before | After | Fat and Oil |
| Name | Condition | Anaerobic | Anaerobic | Anaerobic | Anaerobic | Composition |
| 3-1 | Addition of C | 43 | 7 | 20 | 52 | B |
| | source 1 h | | | | | |
| | before anaerobic | | | | | |

The addition of glucose 1 hour before anaerobic treatment resulted in a greatly improved fat and oil content, compared with Sample 1-1, with the fat and oil content being 52% and the qualitative evaluation of fat and oil composition being B, and also had an improvement effect of fat and oil composition.

This is attributed to that the anaerobic fermentation capability was recovered even by the addition of the carbon source.

### Example 3

### Examination on Addition of Nitrogen Source and Carbon Source

### (1) Preculture, Nitrogen Starvation Culture, and Anaerobic Treatment

It was found from the results of Comparative Example 1 that the nitrogen starvation culture period is enough to be within 48 hours in the view of carbohydrate accumulation.

It was also found from the results of Example 1 and Example 2 that the anaerobic fermentation capability of Euglena cells is restored by adding the nitrogen source or the carbon source before anaerobic treatment.

In this example, further, the following experiment was carried out as Example 3 to examine how simultaneous addition of the nitrogen source and the carbon source affects the fat and oil content and the fat and oil composition.

The conditions of preculture, nitrogen starvation culture and anaerobic treatment were the same as in Comparative Example 1.

### (2) Addition of Nitrogen Source and Carbon Source

Sample 4-1 was prepared by adding glucose as the carbon source in an amount of 1 g per L of culture liquid and diammonium hydrogenphosphate ((NH₄)₂HPO₄) as the nitrogen source in an amount of 0.1643 g (corresponding to 10 mg/L) per L of culture liquid, at 48 hours into the nitrogen starvation culture, which is 0 hour before anaerobic treatment.

Sample 4-2 was prepared by adding glucose as the carbon source in an amount of 1 g per L of culture liquid and diammonium hydrogenphosphate ((NH₄)₂HPO₄) as the nitrogen source in an amount of 0.1643 g (corresponding to 10 mg/L) per L of culture liquid, at 47.5 hours into the nitrogen starvation culture, which is 0.5 hour before anaerobic treatment.

Sample 4-3 was prepared by adding glucose as the carbon source in an amount of 1 g per L of culture liquid and diammonium hydrogenphosphate ((NH₄)₂HPO₄) as the nitrogen source in an amount of 0.1643 g (corresponding to 10 mg/L) per L of culture liquid, at 47 hours into the nitrogen starvation culture, which is 1 hour before anaerobic treatment.

Sample 4-4 was prepared by adding glucose as the carbon source in an amount of 1 g per L of culture liquid and diammonium hydrogenphosphate ((NH₄)₂HPO₄) as the nitrogen source in an amount of 0.1643 g (corresponding to 10 mg/L) per L of culture liquid, at 36 hours into the nitrogen starvation culture, which is 12 hour before anaerobic treatment.

(3) Quantitative Evaluation of Carbohydrate, Extraction and Quantitative Evaluation of Fat and Oil, and GPC analysis

These analyses were performed in the same conditions as in Comparative Example 1.

The fat and oil contents before and after anaerobic treatment are shown in Table 6.

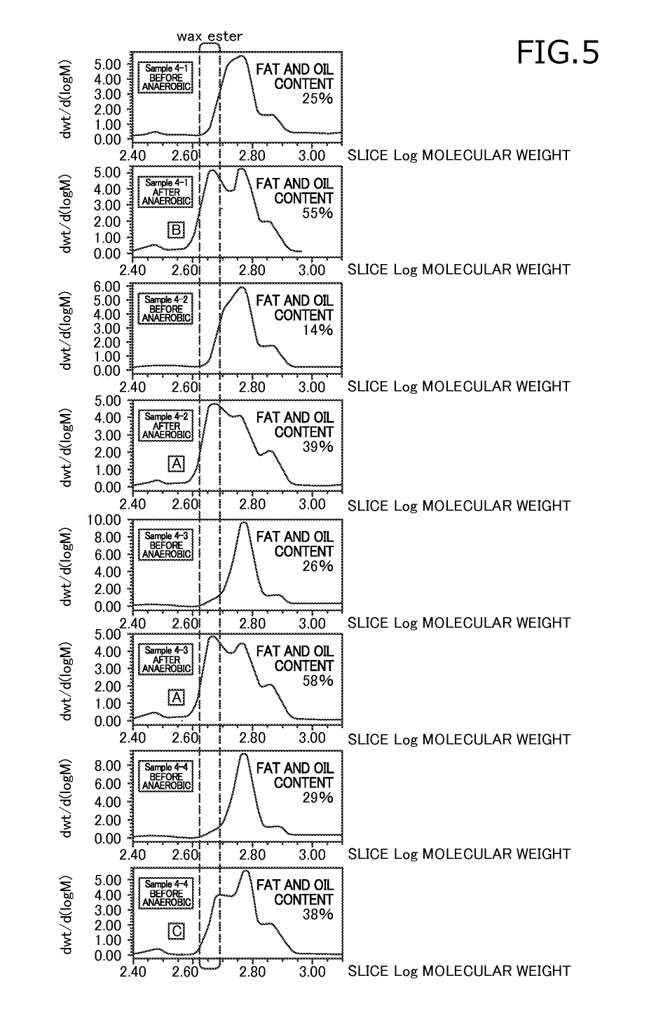

The result of GPC analysis is also shown in FIG. 5.

TABLE 6

| | | Carbohydrate | | Fat and Oil | | Qualitative |
|---|---|---|---|---|---|---|
| | | Content [%] | | Content [%] | | Evaluation of |
| Sample | Examination | Before | After | Before | After | Fat and Oil |
| Name | Condition | Anaerobic | Anaerobic | Anaerobic | Anaerobic | Composition |
| 4-1 | Addition of C | 56 | 5 | 22 | 57 | B |
| | source + N | | | | | |
| | source 0 h | | | | | |
| | before anaerobic | | | | | |
| 4-2 | Addition of C | 40 | 10 | 14 | 39 | A |
| | source + N | | | | | |
| | source 0.5 h | | | | | |
| | before anaerobic | | | | | |
| 4-3 | Addition of C | 23 | 2 | 26 | 58 | A |
| | source + N | | | | | |
| | source 1 h | | | | | |
| | before anaerobic | | | | | |
| 4-4 | Addition of C | 28 | 5 | 29 | 38 | C |
| | source + N | | | | | |
| | source 12 h | | | | | |
| | before anaerobic | | | | | |

The additions of glucose and diammonium hydrogenphosphate 0 to 1 hour before anaerobic treatment resulted in improved fat and oil contents, respectively, compared with Sample 1-1, and also had improvement effects of fat and oil composition (refer to Samples 4-1 to 4-3 in Table 6).

In Sample 4-3, the fat and oil content was improved more greatly than the simple addition of glucose and the addition of diammonium hydrogenphosphate, with the fat and oil content being 58% and the qualitative evaluation of fat and oil composition being A, and the fat and oil composition was also greatly improved.

On the other hand, the addition of glucose and diammonium hydrogenphosphate 12 hours before anaerobic treatment had no effect to improve the fat and oil content and the fat and oil composition, with the result thereof being in the same range as Sample 1-1 (refer to Sample 4-4 in Table 6).

It is supposed that the addition of the carbon source, in addition to the nitrogen source, has a synergistic effect on the improvement in fat and oil content and the improvement in fat and oil composition.

As described so far, in the present invention, as a result of the earnest studies on the problem in which long-time exposure to nitrogen-starved state causes a reduction in wax ester production, it was verified that when a nutrient is added before anaerobic fermentation, the fermentation efficiency is restored, and the wax ester production is improved.

It was found that the nutrient being added can be a carbon source although a nitrogen source is preferred.

It was further verified that simultaneous addition of the nitrogen source and the carbon source leads to further improvement in fat and oil composition, compared with independent additions of these sources.

When the nutrient is added, the decomposition of the accumulated paramylon is promoted since the nitrogen starvation state is resolved thereby.

Thus, if the timing of adding the nutrient is too early, the nitrogen starvation state is restored due to the consumption of the added nutrient during culture.

Therefore, tests were performed as described above, whereby it could be confirmed that the timing of adding the nutrient is 3 hours before anaerobic fermentation, more preferably within 1 hour before anaerobic fermentation.

In addition to the above-mentioned conditions, the amount of the nutrient being added is also important.

An excessively large addition amount results in a reduced accumulation amount of paramylon, and an excessively small addition amount does not lead to the improvement in anaerobic fermentation efficiency.

Therefore, in the present invention, diammonium hydrogenphosphate is added so that the ammonium ion concentration becomes about 10 mg/L.

This amount of 10 mg/L is an amount to be consumed for 6 to 7 hours since the consumption rate of ammonia is about 1.5 g·L⁻¹·h⁻¹ when Euglena concentration is about 0.3 g/L.

The wax esters thus improved in production can be effectively used as biofuels.

Euglena is a readily available microorganism as used also for health foods and the like, and can be cultured in large amounts.

According to the present invention, clean energy can be stably supplied by recovering a large amount of good-quality wax esters from Euglena that is such a microorganism.

1. A method for producing Euglena having a high wax ester content, comprising:
performing at least a first step of aerobically culturing microalga Euglena under nitrogen starvation conditions and a second step of holding the cells in an anaerobic state, and
adding, before the second step, a nutrient source to the culture liquid which has passed through the first step.

2. The method for producing Euglena having a high wax ester content according to claim 1, wherein the addition of the nutrient source is performed at earlier timing based on the point of time at which the dissolved oxygen concentration of the culture liquid in the anaerobic state falls below 0.03 mg/L in the second step.

3. The method for producing Euglena having a high wax ester content according to claim 1, wherein the nutrient source is a nitrogen source.

4. The method for producing Euglena having a high wax ester content according to claim 1, wherein the nutrient source is a carbon source.

5. The method for producing Euglena having a high wax ester content according to claim 1, wherein the nutrient source is composed of a nitrogen source and a carbon source.

6. The method for producing Euglena having a high wax ester content according to claim 3, wherein the nitrogen source is an ammonium compound.

7. The method for producing Euglena having a high wax ester content according to claim 5, wherein the nitrogen source is an ammonium compound.

8. The method for producing Euglena having a high wax ester content according to claim 4, wherein the carbon source is glucose.

9. The method for producing Euglena having a high wax ester content according to claim 5, wherein the carbon source is glucose.

10. The method for producing Euglena having a high wax ester content according to claim 2, wherein the nutrient source is a nitrogen source.

11. The method for producing Euglena having a high wax ester content according to claim 2, wherein the nutrient source is a carbon source.

12. The method for producing Euglena having a high wax ester content according to claim 2, wherein the nutrient source is composed of a nitrogen source and a carbon source.

13. The method for producing Euglena having a high wax ester content according to claim 10, wherein the nitrogen source is an ammonium compound.

14. The method for producing Euglena having a high wax ester content according to claim 12, wherein the nitrogen source is an ammonium compound.

15. The method for producing Euglena having a high wax ester content according to claim 11, wherein the carbon source is glucose.

16. The method for producing Euglena having a high wax ester content according to claim 12, wherein the carbon source is glucose.
